(19) United States
(12) Patent Application Publication
(10) Pub. No.: US 2019/0262002 A1
(43) Pub. Date: Aug. 29, 2019

(54) NOVEL ENHANCED ORB-LIKE INTRASACULAR DEVICE

(71) Applicant: NEURVANA MEDICAL LLC, Tustin, CA (US)

(72) Inventor: Joshua BENJAMIN, Mission Viejo, CA (US)

(73) Assignee: NEURVANA MEDICAL LLC, Tustin, CA (US)

(21) Appl. No.: 16/284,877

(22) Filed: Feb. 25, 2019

Related U.S. Application Data
(60) Provisional application No. 62/634,712, filed on Feb. 23, 2018.

(51) Int. Cl. A61B 17/12

(52) U.S. Cl. CPC A61B 17/12113; A61B 2017/00867; A61B 17/12031; A61B 17/12168

(57) ABSTRACT

Aneurysm embolization devices made from contiguous superelastic shape-memory material that self-expands for intrasaccular aneurysm treatment.

### CROSS-REFERENCE TO RELATED APPLICATIONS

This application claims priority from U.S. Prov. App. Ser. No. 62/634,712, filed Feb. 23, 2018, the contents of which is incorporated herein by reference in its entirety.

### BACKGROUND

The pathological course of a blood vessel that is blocked is a gradual progression from reversible ischemia to irreversible infarction (cell death). A stroke is often referred to as a “brain attack” and occurs when a blood vessel in the brain becomes blocked or ruptures. An ischemic stroke occurs when a blood vessel in the brain becomes blocked. Occlusions may be partial or complete, and may be attributable to one or more of emboli, thrombi, calcified lesions, atheroma, macrophages, lipoproteins, any other accumulated vascular materials, or stenosis. Ischemic strokes account for about 87% of all strokes. Hemorrhagic strokes, which account for the remaining 13% of strokes, occur when a blood vessel in the brain ruptures. Stroke is the third leading cause of death in the United States, behind heart disease and cancer and is the leading cause of severe, long-term disability. Each year roughly 795,000 Americans experience a new or recurrent stroke. Stroke is the number one cause of inpatient Medicare reimbursement for long-term adult care. Total stroke costs now exceed $34 billion per year in U.S. healthcare dollars. An occlusion in the cerebral vasculature can destroy millions of neurons and synapses of the brain.

An aneurysm is a bulging of a blood vessel wall that is abnormal. Saccular aneurysms appear like a sac protruding out from a parent vessel, have a neck and can be prone to rupture. Fusiform aneurysms involve a blood vessel expanded circumferentially in all directions. Fusiform aneurysms generally do not have a neck and are less prone to rupturing than saccular aneurysms. As an aneurysm grows larger, the walls forming it generally become thinner and weaker. This decrease in wall strength, particularly for saccular aneurysms, increases the risk of the aneurysm rupturing and releasing blood into the surrounding tissue. Serious and potentially fatal health outcomes are typical associated with these conditions. Cerebral aneurysms are aneurysms that occur in the intercerebral arteries that supply blood to the brain. The majority of cerebral aneurysms form at the junction of arteries at the base of the brain.

Although identification of intact aneurysms is increasing due to improved medical imaging technologies and use thereof, often cerebral aneurysms remain undetected when existing in unruptured form. When an aneurysm ruptures, it often causes stroke, disability, and/or death. The prevalence of cerebral aneurysms is generally estimated to be in the range of 4% of the general population. Approximately 30,000 people per year suffer a ruptured cerebral aneurysm in the U.S. alone. About 10% with aneurysm hemorrhage die before reaching medical attention, 25% die within 24 hours, 40-49% die within 3 months, and mortality has been estimated to be as high as 65%, with most deaths occurring early in the clinical course.

Aneurysm treatment varies depending on a variety of factors and includes both surgical and non-surgical methods and devices such as blood pressure control, clipping and coiling. Each has attendant risks and varied clinical outcome. The presently disclosed devices and methods address these and other needs in the art.

### SUMMARY

The present devices relate to expandable endovascular treatment devices. As described herein intrasacular devices are provided comprised of nickel-titanium alloys (nitinol). The compliance, shape memory, rapid low force deployment, and tuned stiffness of the presently described orb-like scaffolding devices permits addressing acute states of aneurysm to achieve improved clinical outcomes. The particularly disclosed embodiments are provided in unique shapes, providing device shape compliance levels heretofore not seen in the art. The orb-like shaped intrasacular device embodiments described herein are often combined with other therapies or used alone, such as with coils, flow diverters and the like, in the treatment of a subject.

According to embodiments there is disclosed a system for treatment of aneurysms which comprises an orb-like scaffolding device, often comprised of nitinol, which is soft, compliant and conformable, ranging in an expanded size from at least at or about 1.5 mm to at or about 12 mm and deliverable through a microcatheter delivery device. In certain embodiments the expanded width of an expanded orb-like scaffolding device ranges from about 1.5 mm to about 6 mm, and is deliverable through a 0.4 mm microcatheter, or between 0.25 mm to about 0.4 mm. In certain other embodiments the expanded width of an expanded orb-like scaffolding device ranges from about 6.5 mm to about 12 mm, and is deliverable through a 0.5 mm microcatheter, or between 0.5 to about 0.6 mm. In frequent embodiments, the delivery device comprises a 10MC, 18MC, or 21MC. The inner diameter of microcatheter can range, for example, from 0.25 mm to at or about 0.6 mm. When used according to presently contemplated methods the orb-like scaffolding devices are used to treat an aneurysm size of between about 3 mm to about 12 mm. Often, the aneurysm size treated or treatable using the presently described devices is between about 8 mm to about 15 mm.

In frequently included embodiments, an intrasaccular occlusion device is provided for addressing intravascular aneurysms, comprising a contiguous scaffold, wherein the scaffold is defined by zones of flexure and open cells and is self-expanding between positions defined by a compressed position and an expanded position, the expanded position defining an orb-like shape; wherein the expanded position of the scaffold is between at or about 1.5 mm to at or about 12 mm in diameter; and wherein the compressed position is between about 0.25 mm to about 0.6 mm in diameter. Often, the scaffold is formed of nickel-titanium alloy (nitinol). Also often, the contiguous nitinol scaffold is formed of a single material, without breaks or welds. Most frequently in the embodiments described herein, the scaffold is laser cut from a single nitinol tube. Often, the open cells are sized to permit passage of an embolic coil therethrough. Also often, the scaffold is radiopaque, or comprises a radiopaque marker. In frequently included embodiments, the device is adapted to be placed in a microcatheter having an inner diameter of between about 0.25 mm to about 0.6 mm. Often, the expanded position is between about 1.5 mm to about 6 mm, and the compressed position is between about 0.25 mm to about 0.4 mm. Also often, the expanded position is between about 6.5 mm to about 12 mm, and the compressed position is between about 0.5 mm to about 0.6 mm.

Also in frequently included embodiments, a self-expanding intrasaccular occlusion device formed of a single, contiguous superelastic shape-memory material is provided comprising a scaffold, wherein the scaffold is defined by zones of flexure and open cells and is self-expanding between positions defined by a compressed position and an expanded position, wherein the expanded position has a diameter of between about 16 to about 26 times the size of the diameter of the compressed position. Often, the single, contiguous superelastic shape-memory material comprises a nitinol scaffold laser cut from a single nitinol tube. Also often, the expanded position of the scaffold is between at or about 1.5 mm to at or about 12 mm in diameter; and the compressed position is between about 0.25 mm to about 0.6 mm in diameter. In often included embodiments, a system is provided comprising the self-expanding intrasaccular occlusion device of claim 10, further comprising a guidewire and deployment anchor.

In frequent embodiments the device is orb-shaped in the expanded position. Also frequently, the device is mushroom-, bullet- or funnel-shaped in the expanded position.

In frequent embodiments, a method of making an intrasaccular occlusion device is provided involving forming a single, contiguous superelastic shape-memory material into to a predefined pattern, the pattern defining a scaffold having zones of flexure and open cells; optionally or specifically processing the superelastic shape-memory material to comprise the scaffold having an orb-like shape memory; and optionally or specifically finishing the scaffold to be biocompatible. Often, the material of the device is nitinol. In frequent embodiments, the nitinol comprises a single nitinol tube and the forming step comprises laser cutting the nitinol tube according to the predefined pattern. In frequent embodiments, the zones of flexure and open cells are placed in a manner within the scaffold that when in an orb-like shape it is flexible, soft and compliant. Often the methods of making include further steps involving testing to ensure compliance with quality control needed for neurovascular placement.

In certain other embodiments the single, contiguous superelastic shape-memory material is formed by additive manufacture. And, in such embodiments, the material often comprises nitinol.

In frequent embodiments of the present disclosure, methods of treating aneurysm are provided, involving positioning a device of the type described herein adjacent the neck of an aneurysm; deploying the device within the sac of the aneurysm and permitting the device to self-expand within the sac; and introducing an embolic material to the sac and within the device through one or more of the open cells.

In also included embodiments, a laser cut nitinol device, is provided having orb-like or funnel shape ranging in size from at least about 2 mm to around 10 mm; and deliverable through a 0.017 catheter and devices having smaller profiles; whereby the same may be used alone or with microcoils. According to frequently included embodiments, the devices are more soft, compliant and conformable than conventional laser cut articles used for minimally invasive surgical interventional procedures. Often, use of systems comprising such devices improve aneurysm neck support and coverage over conventional devices in situ. Often, the devices are used in complement with embolic coils, wherein the number of coils deployed is less than or equal to the number used in conventional procedures. Often, the devices are compatible with platinum coils and do not require anti-coagulant therapy. In frequent embodiments, the devices are radiopaque and can be visualized during and post-procedures. Also frequently, the devices can be manufactured at scale. The laser cut nitinol tube is provided with a pattern that allows it to be more flexible, soft and compliant than expected when emplaced. Often zones of flexure permit the laser cut nitinol tube to be expanded from a first to a second position with the brain, for emplacement within the sac of an aneurysm, without any insult or injury to the surrounding vasculature. In use, unexpectedly difficult placements are achieved within the brain of a patient, whereby fewer complementary devices, such as coils are used. In certain embodiments, the laser cut nitinol tube is able to be emplaced through a delivery catheter having a profile less than or equal to 0.017. The devices may move from a first, retracted, to a second, expanded configuration. The devices may also emanate from a flattened section during the first configuration. In frequent embodiments, the devices are expandable and compressible in a second configuration on to be used to pack an aneurysm. In frequently included embodiments, the devices are adequately compliant to be used in multiple versions, along or with other therapies, in situ. In use such devices are effective to counter the growth of aneurysms in the brain. In certain embodiments, the scaffold is comprised of a bioresorbable, anti-infective, and/or biodegradable material, and/or are fully compostable.

According to embodiments, there are disclosed the systems, devices, methodologies of manufacture, deployment and quality control used in advancing the intrasacular devices into patients; and methods of making the described intrasacular devices.

### BRIEF DESCRIPTION OF THE DRAWINGS

FIG. 1 shows an embodiment of an intrasacular occlusion device of the present disclosure.

FIG. 2 shows an exemplary template for manufacturing an intrasaccular occlusion device of the present disclosure by laser cutting.

FIG. 3 shows a schematized device with operative and novelty holding zones of flexure, reinforcement elements and flattened distal end according to this filing.

FIG. 4 shows another embodiment of an intrasacular occlusion device of the present disclosure.

FIG. 5 shows another view of the embodiment of an intrasacular occlusion device of the present disclosure depicted in FIG. 4.

### DETAILED DESCRIPTION

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, the term “and/or” may mean “and,” it may mean “or,” it may mean “exclusive-or,” it may mean “one,” it may mean “some, but not all,” it may mean “neither,” and/or it may mean “both.”

As used herein, the term “subject” is not limited to a specific species. For example, the term “subject” may refer to a patient, and frequently a human patient. However, this term is not limited to humans and thus encompasses a variety of mammalian species.

As used herein, a “delivery device” or “delivery system” refers to a microcatheter or catheter system known in the art adapted to permit placement and extraction of the presently described devices to/from the location of an aneurysm.

As used herein, the term “scaffold” refers to an interconnected matrix support structure defining a plurality of open cells, often comprised of nitinol or other material having super elastic and/or shape memory traits. Presently contemplated scaffolds for the orb-like devices described herein utilize the interconnected matrix support structure defining a plurality of open cells, for example, to tune stiffness and compliance of the devices with the greatest structural integrity in 360° while permitting the devices to be compressed into the smallest possible form for accurate and less traumatic delivery, and permit fluid passage therethrough.

As used herein, “scaffold device” refers to a device contemplated herein formed of a scaffold material, often nitinol, and may be orb-like in shape, or another shape noted herein.

As used herein, the term “orb” refers to an orb shape. The terms “orb-like” also refers to a shape related to the shape of an orb. As used herein, unless specifically states otherwise, orb-like as used herein refers to circular, generally circular, mushroom-shaped, bullet-shaped, and related shapes.

As used herein, “delivery device” refers to a device such as a catheter or similar devices known in the art for use in delivery and at least part of the placement procedure of the presently described devices in a subject at a delivery site or detachment location. A guidewire, a pusher, and related hardware are intended to be included in this definition.

In terms of global populace, the largest growing demographic currently unaddressed and needing to be managed are those having strokes, or brain events based upon transient or permanent occlusion events within the relevant and proximate vasculature to that of the brain. Since embolic coils are state of the art for brain aneurysm treatment, various approaches have tried to alter these devices and procedures with limited success. The presently described devices may be used alone or together as a complement to existing embolic treatments in such way as clinicians, physicians and surgeons determine to align with optimal patient care.

The present disclosure relates to neurovascular medical systems of treatment, devices, methods and approaches to manufacturing devices involved in the same. More specifically, the devices of the present disclosure are used to intervene and solve acute issues, as well as long term aneurysm treatments, alone or in combination with embolic coils and other related tools of the clinician. The present devices provide an intrasacular scaffolding for use in, for example, wide neck aneurysms, which drastically reduces or eliminates the need for dual antiplatelet regimen typically associated with embolism treatment.

Certain existing devices contemplate the use of a graft or stent and foreign objects to repair an aneurysm. In such a system, the foreign objects are placed within the aneurysm sac and the graft/stent is placed and is intended to retain the foreign object within the sac as well as to provide a passage for fluid flow. Due to the complexity of such systems, it is necessary to coordinate the sequence of deployment of the various components of the system over time. Alternatively, in the event the graft or stent is placed prior to the foreign objects in the aneurysm, it must include sidewall openings permitting the foreign objects to be added within the aneurysm sac. Other concepts in the art require much large delivery devices than are capable of use in conjunction with the presently described orb-like scaffolding, and provide a notably non-uniform structure with their device structure that is both uneven in their deployment and difficult to retrieve or return to a catheter.

The presently described orb-like scaffolding devices provide for a low delivery force using existing delivery platforms. This delivery force is lower than stent retrievers and on-par with or slightly higher than coils.

The present disclosure provides a device to be used, for example, in the cerebral vasculature to achieve desired clinical outcomes in a repeatable and scalable fashion. Addressing aneurysms may be done with the present invention alone or in combination with coils and other devices. In accordance with use with complementary devices, a clot can be addressed in-situ to reperfuse a blood vessel without occluding or blocking blood flow and without requiring the use of additional structures to address distal embolization, while an aneurysm exists to allow progressive treatments.

The presently described intrasaccular devices can be configured to be deployed in the cerebral arteries, including but not limited to: the anterior cerebral arteries (ACA), the anterior communicating artery, the middle cerebral arteries (MCA) (including the M1 and M2 segments), the posterior communicating arteries, the internal carotid arteries (ICA), the vertebral arteries, the basilar artery, and the posterior cerebral arteries (PCA). Other embodiments of the disclosure are not limited to use in the neurovasculature and may be used in other regions, including but not limited to vessels (e.g. veins or arteries) in, to or from the heart, lungs, extremities (e.g., legs), and pelvis.

Delivery systems (also referred to as a category of complementary devices) that can be utilized in connection with the orb-like devices described herein include delivery catheters in present use. For example, U.S. Pat. Nos. 8,070,791; 8,926,680; 8,945,143; 8,574,262; 9,198,687; 9,220,522; and/or 8,088,140 describe certain examples of contemplated type of intravascular delivery devices. The presently described orb-like devices are compatible with multiple coil pusher platforms, and may be used in a complementary manner with intrasaccular occlusion devices such as those described in U.S. patent application Ser. No. 15/755,071, filed Feb. 24, 2018 (not yet published) and PCT Pub. No. WO2017035275, coils, or complementary to intralumenal devices.

In frequently included embodiments, a method of treating an aneurysm is provided. An orb-like scaffolding device of the present disclosure is positioned in a catheter delivery device in a manner permitting its deployment through axial pressure exerted on its proximal anchor region. The orb-like scaffolding device is then advanced to the location of the aneurysm. In one embodiment, the location of the aneurysm refers to the location of the neck of the aneurysm. Advancing the delivery device to the location of the aneurysm can also mean advancing the delivery device to within or to the side of the aneurysm, depending on the location and morphology of the aneurysm. The orb-like scaffolding device is generally delivered through a delivery device so that the self-expanding orb-like scaffolding remains in a non-expanded configuration until a desired location is reached. The orb-like scaffolding device can then be advanced out of the delivery device, or the delivery device (or portion thereof) is withdrawn until the distal end of the self-expanding orb-like scaffolding is aligned with, or slightly distal to, the distal end of the microcatheter. Thereafter the movement the self-expanding orb-like scaffolding is continued to permit the scaffolding to expand within the aneurysm. Often the placement of the orb-like scaffolding is guided by a visualization and/or guiding technology. Also, according to the presently contemplated methods, the orb-like scaffolding may be partially released from the delivery device and then the delivery device is adjusted to better position the orb-like scaffolding within the aneurysm. In such embodiments, the orb-like scaffolding may be fully reintroduced to the delivery device prior to adjustment.

As noted, in presently contemplated embodiments, the delivery device is often retracted proximally, thereby unsheathing the self-expanding orb-like scaffolding and allowing the self-expanding scaffolding to deploy to its expanded configuration within the aneurysm.

In several embodiments, after a period of time after initial expansion of the self-expanding scaffold, the delivery device can be advanced proximally to compress and resheath the self-expanding orb-like scaffolding and then the delivery device can be retracted distally again to redeploy the orb-like scaffolding in the same or different position. The resheathing and unsheathing can be repeated one or more times. The orb-like scaffolding device can then be removed by advancing the delivery device distally to compress and resheath the orb-like scaffolding and then withdrawing the orb-like scaffolding from the body (with or without concurrent removal of the delivery device).

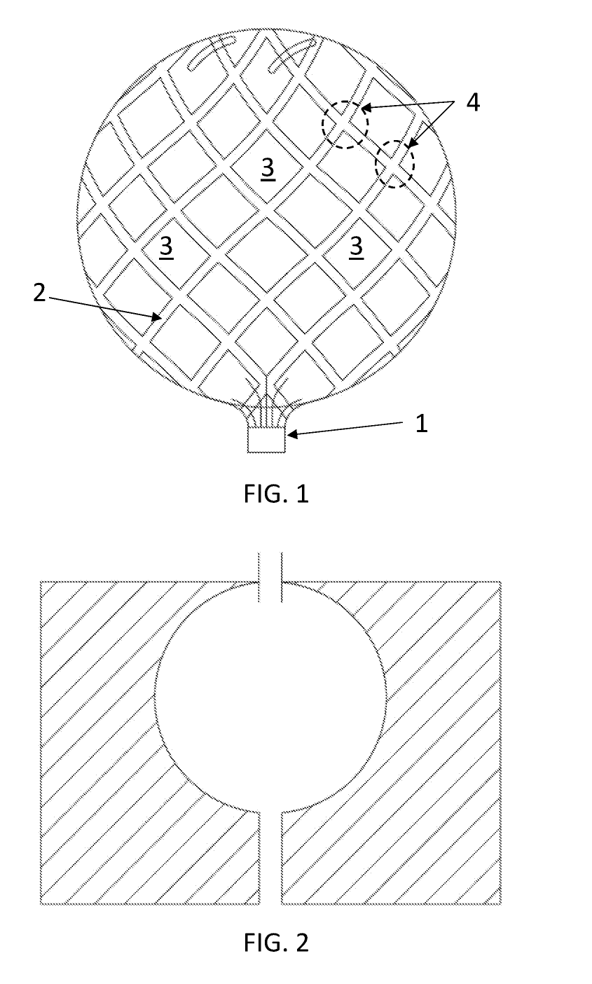

FIG. 1 shows a pattern for an exemplary orb-like scaffold device of the present disclosure. Such devices are created, for example, via laser cutting of nitinol. The matrices of the present devices are made to be compliant with the walls of the vessels in which they are emplaced. The presently disclosed devices are characterized as soft, such that the scaffolding flexes under low external pressure to aid in the matrices adaptation and compliance.

FIG. 2 shows a schematized device with device for creating the geometry including unique zones of flexure, according to this filing, with the darkened bands indicating edges and of respective zones, allowing for an Orb-like arrangement and folding of the device for delivery.

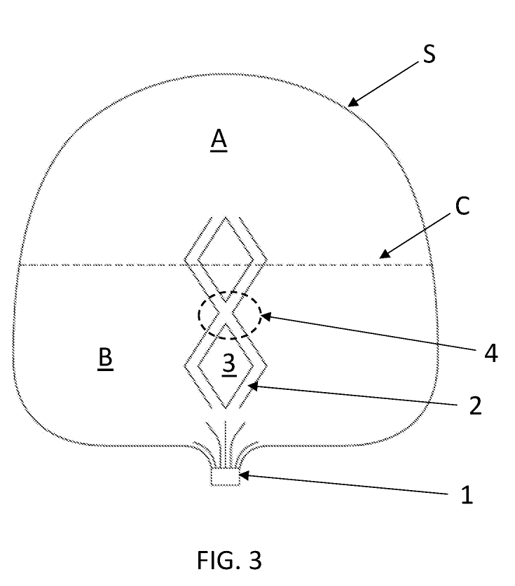

FIG. 3 likewise shows features and shapes of devices according to the instant teachings, details shown of junctures and flex points (zone of flexure 4), that is adapted to be loaded and mounted for delivery in the lumen of a microcatheter, along with alignment, strengthening and attachment. Reference S refers to the general shape of the exemplary device, and in this regard “orb-like” is intended to refer to mushroom and bullet shapes, in addition to circular shapes.

Also in reference to FIG. 3, in a separate embodiment, a self-expanding scaffold device is provided that comprises the shape identified in area A, above reference C. Such an embodiment would not include the area of reference B. Such a dome shape may resemble a rosebud or funnel and contain the scaffold structure described for other device embodiments described herein. The upper portion of such embodiments would be placed within the sac of the aneurysm. The area across reference C, in such embodiments, would be preferably flat and open and when inserted into the aneurysm sac would face the parent vessel. The placement of the open end of such embodiments might, for example, lie flush or immediately adjacent the parent vessel and within the aneurysm sac. Such embodiments are deliverable in a similar manner to that described herein and adaptable for delivery in delivery devices having inner lumen sizes similar to or the same as embodiments of the of orb-like scaffold device embodiments.

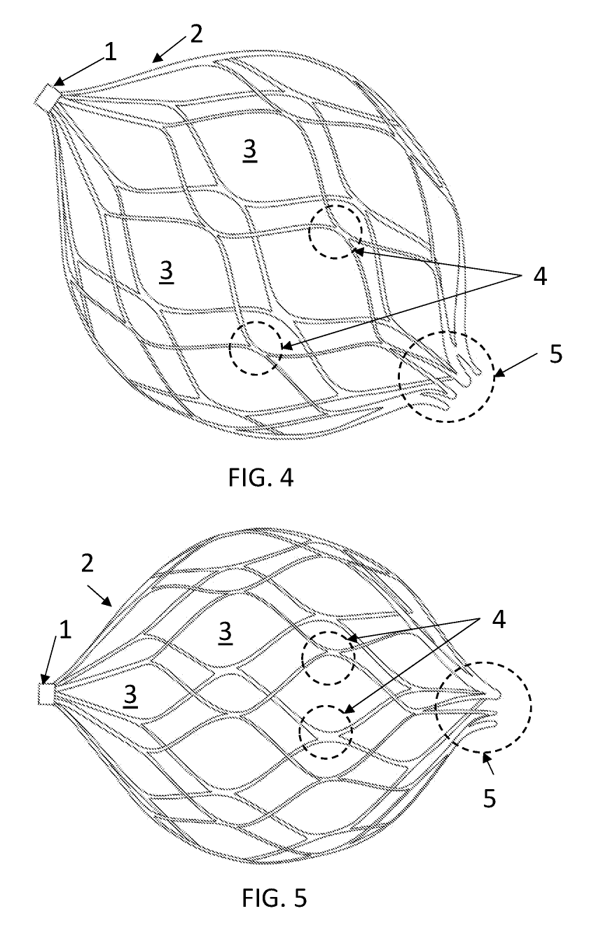

FIGS. 4 and 5 show different views of an embodiment of an exemplary orb-like scaffold device of the present disclosure. The embodiment shows cell structures 3 defined by the scaffold/matrix material 2 that allow for rapid deployment, correct placement, and effective occlusion use. The distal end 5 of the orb-like scaffolding is depicted as pointed distally for illustrative purposes only. In the most frequent presently included embodiments, the distal end 5 of the present devices is generally flat or curved inward toward the interior of the expanded scaffold. A proximal deployment anchor 1, is included and acts as a link to pusher within and out of the delivery device. The deployment anchor is often adapted to be utilized with a variety of delivery devices, though a flat cylindrical deployment anchor is depicted. The detachment mechanism can be provided using a variety of detachment technologies known in the art, including mechanical wire release, dissolving of wire (electrolytic detachment), melting of thermal plastic. In each case, the detachment mechanism utilized holds the scaffold device at the deployment anchor until a predetermined or desired detachment location is obtained.

The cell openings in the scaffold not only provide for the remarkably large self-expansion, flexibility and softness of the presently described scaffold devices, but they also permit the passage of embolic material such as embolic coils therethrough when the scaffold device is placed in the sac of an aneurysm.

FIGS. 4-5 each show manufactured embodiments of an exemplary orb-like scaffold device of the present disclosure. The depicted device was laser cut from a single laser-cut nitinol tube. As described also herein, this device as with others described herein within the scope of the present disclosure is, in use, deployed into an aneurysm sac for occlusion of the aneurysm.

While nitinol is flexible and super elastic under the presently contemplated use conditions, the scaffolding 2 comprising the orb aspect of the device is adapted to contain zones of flexure 4. These zones are defined by the cells of the orb-like scaffolding, as cane be seen in FIGS. 4-5. Areas of varying stiffness or compliance are thereby provided such that outside a zone of flexure 4 the orb-like scaffolding is less compliant relative to an area within a zone of flexure. Moreover, different stiffness levels between zones of flexure 4 in an orb-like scaffolding are often provided in certain embodiments. Often the cell 3 pattern defined by the structural material in the scaffolding is characteristic of the compliance of the device. The orb-like scaffolding 2 is often flex-tuned to provide a predetermined and optimal balance of compliance and stiffness for a variety of purposes. Some of these purposes include, for example reducing the minimum compacted size achievable for placement in a delivery catheter, providing a large achievable delta in size between compacted and deployed states, and accurate emplacement. In the tuned scaffolding 2, radial outward force can be adapted or tuned by varying stiffness or compliance using determined placement locations of zones of flexure 3. The scaffold of the Orb-like device is adapted to be compressed, flattened, folded, or otherwise reduced in circumference when placed under outward radial pressure or contacted in a predetermined manner into a “delivery shape” within a delivery port of a catheter for placement.

In certain frequent embodiments, the delivery shape of the scaffolding devices described herein resembles the shape of the nitinol tube from which it was laser cut (the source tube) and is often about the same, or the same, diameter as the source tube. Thus, particularly small delivery devices are useful for the present devices, despite their relatively larger self-expanded form. Compliance relative to outward radial force is managed, for example, with the scaffold structure. Upon deployment and release from the delivery device, the orb-like scaffolding expands to a predetermined orb-like shape. The environment in which it is deployed and the purpose of the scaffolding often guides the shape of the scaffolding. For example, since the scaffold devices of the present disclosure are generally soft and compliant, they will often assume the specific shape of the aneurysm sac into which they are positioned.

The orb-like scaffolding provides a structure with 360 degrees of structural integrity in any plane. Thus, a sphere of contiguously interlaced matrix material having zones of flexure and defining open cells is provided by the presently described orb-like scaffolding devices. This sphere of contiguously interlaced matrix material having zones of flexure and defining open cells is useful to aid emplacement of the devices and ensuring that they stay where they are placed, with a uniform pressure and/or low level of stress exerted on the aneurysm wall. Uniform pressure 360 degrees around the interior wall of an aneurysm is provided by frequent embodiments of the present devices. This uniform pressure and/or low level of stress exerted on the aneurysm wall reduces the likelihood that the aneurysm wall will be damaged upon or after emplacement by the device. This differs from the existing art in many respects in that they do not provide the full scope of structural integrity of the present devices and they do not provide uniform pressure and/or low stress inside an aneurysm sac.

In the operation of the devices of the present disclosure, often it is understood that often no anti-coagulant therapy is needed. Nevertheless, at the discretion of the physician such therapy may be provided or recommended. Moreover, the presently described devices permit the treatment of amorphous and hard to manage aneurysm necks.

In certain embodiments, after placement of orb-like scaffolding device of the present disclosure in an aneurysm, coils, embolic material, or other foreign material or reagents may be introduced within the cells in the orb-like scaffolding, for example, to aid in clotting the area.

nitinol, as noted, is one exemplary category of specifically suitable material used for the contemplated devices. In the frequent embodiments, nitinol is laser cut according to a template and formed into the presently described devices. While other methods of nitinol cutting and shaping are contemplated, laser cutting is frequently preferred due to its ease of use, the precision of the resulting cut, and preservation of the integrity of material adjacent to the cut. Moreover, laser cutting eases manufacture of the presently described devices.

In the most frequently included embodiments, the scaffold devices of the present disclosure are formed from a single contiguous nitinol tube. In this regard, the expanded matrix comprises, in the most frequently included embodiments, a single contiguous material, without welding or attachments to hold the scaffold matrix material together or form it into a specific shape. Nevertheless, a deployment anchor may comprise an added element introduced to the contiguous scaffold matrix at its proximal end or it may be formed of the same material. In such embodiments, the tube is laser cut according to a predetermined pattern. Thereafter, the cut nitinol matrix is induced through methods known in the art of cutting and shaping nitinol to have a predetermined orb-like shape memory. This orb-like shape is expanded in relative diameter versus the nitinol tube from its source tube. Often the self-expanded scaffold of the orb-like scaffold devices described herein is in certain embodiments about 16 to about 26 times the diameter of the source tube.

Other methods of production, design and manufacture are contemplated, including additive manufacture. For example, orb-like scaffolding devices of the present disclosure comprised of nitinol are manufactured using additive manufacture according to known methods using appropriate concentrations of nickel and titanium powder. See, e.g., Walker et al., ASME 2014 International Mechanical Engineering Congress and Exposition, Vol. 2A (2014). Selective laser melting, electron Beam Melting, Fused Deposition Modelling, Wire Arc Additive Manufacturing, and Laser Engineered Net Shaping such as Laser Cladding are some of the contemplated methods of additive manufacture. See Humbeeck, Shape Memory and Superelasticity, Vol. 4, Issue 2, pp. 309-12 (June 2018); Elahinia et al., Prog. Mater. Sci. 83:630-663 (2016). Additively manufactured nitinol is known to be biocompatible, and adjusted via aligned laser processing conditions. See, Habijan et al., Mater. Sci. Eng., 33:419-426 (2013). Other shape memory and superelastic alloys and materials can be similarly additively manufactured and used in the presently described devices and methods. See, e.g., Gustmann et al., Shape Memory and Superelasticity, Volume 3, Issue 1, pp 24-36 (March 2017)

In frequently included embodiments, the cut or manufactured nitinol is treated using known electropolishing, passivation, pickling, or secondary processing techniques to, inter alia, form a stable TiO₂ barrier against ion exchange. In addition, other shape memory and/or superelastic material that is or can be made nontoxic, bioinert, nonallergic, and does not release harmful byproducts also contemplated as suitable within the scope of the instant teachings.

In one exemplary use, the presently contemplated scaffold devices are utilized with neurovascular embolic coils for the treatment of unruptured wide-necked intracranial aneurysms with neck widths of about or greater than 4 mm, or those that have a dome to neck ratio of less than 2, originating on or near a vessel bifurcation of the basilar tip or carotid terminus with at least a portion of the aneurysm neck overlapping the lumen of the parent artery. In frequently contemplated settings, the inflow have diameters of at or about 2.7 mm to at or about 4.5 mm, though sizes may vary. In practice, the scaffold device is provided attached to a delivery wire and used in conjunction with a pusher. As noted, the scaffold devices of the present disclosure are detachment-means agnostic and intended to be used with commercially available detachment power supplies. Exemplary steps of a procedure of using a scaffold device of the present disclosure in a dual catheter setting often include:
1) Preparing the catheter guide device for the intended procedure;
2) Placing the guide device into the appropriate artery;
3) Preparing the microcatheter and guide wire;
4) Using catheterization techniques and fluoroscopic guidance, place the microcatheter proximal to the vessel bifurcation;
5) Removing the guide wire from the microcatheter;
6) Using angiography, determine the aneurysm location, and relevant sac and/or next size information, and parent vessel diameter;
7) Selecting an indicated size scaffold device;
8) Placing an introducer and a scaffold device the into the delivery microcatheter;
9) Advancing the delivery wire and scaffold device until radio-identifiable markers on the scaffold device close to the microcatheter tip;
10) Advancing the scaffold device and microcatheter as a unit until the microcatheter tip is positioned just proximal to the aneurysm neck and at the level of the vessel bifurcation;
11) Pulling back on the delivery wire and microcatheter until the tip of the microcatheter begins to move and extend the distal end of the scaffold device outside the microcatheter; and
12) Positioning the scaffold device so that the its radio-identifiable markers are directly proximal to the aneurysm neck and then continue to deploy the scaffold device within the aneurysm sac.

Groupings of alternative elements or embodiments of the invention disclosed herein are not to be construed as limitations. Each group member may be referred to and claimed individually or in any combination with other members of the group or other elements found herein. One or more members of a group may be included in, or deleted from, a group. Many variations to those methods, systems, and devices described above are possible. Since modifications and variations to the examples described above will be apparent to those of skill in this art, it is intended that this invention be limited only by the scope of the appended claims.

One skilled in the art will appreciate further features and advantages of the presently disclosed methods, systems and devices based on the above-described embodiments. Accordingly, the presently disclosed methods, systems and devices are not to be limited by what has been particularly shown and described, except as indicated by the appended claims. All publications and references cited herein are expressly incorporated herein by reference in their entirety and/or for the specific reason for which they are cited herein. Citation of the above publications or documents is not intended as an admission that any of the foregoing is pertinent prior art, nor does it constitute any admission as to the contents or date of these publications or documents.

1. An intrasaccular occlusion device for addressing intravascular aneurysms, comprising:
a contiguous scaffold, wherein the scaffold is defined by zones of flexure and open cells and is self-expanding between positions defined by a compressed position and an expanded position, the expanded position defining an orb-like shape;
wherein the expanded position of the scaffold is between at or about 1.5 mm to at or about 12 mm in diameter; and
wherein the compressed position is between about 0.25 mm to about 0.6 mm in diameter.

2. The device of claim 1, wherein the scaffold is formed of nickel-titanium alloy (nitinol).

3. The device of claim 2, wherein the contiguous nitinol scaffold is formed of a single material, without breaks or welds.

4. The device of claim 2, wherein the scaffold is laser cut from a single nitinol tube.

5. The device of claim 1, wherein the open cells are sized to permit passage of an embolic coil therethrough.

6. The device of claim 1, wherein the scaffold is radiopaque, or comprises a radiopaque marker.

7. The device of claim 2, wherein the device is adapted to be placed in a microcatheter having an inner diameter of between about 0.25 mm to about 0.6 mm.

8. The device of claim 1, wherein the expanded position is between about 1.5 mm to about 6 mm, and the compressed position is between about 0.25 mm to about 0.4 mm.

9. The device of claim 1, wherein the expanded position is between about 6.5 mm to about 12 mm, and the compressed position is between about 0.5 mm to about 0.6 mm.

10. A self-expanding intrasaccular occlusion device formed of a single, contiguous superelastic shape-memory material comprising a scaffold, wherein the scaffold is defined by zones of flexure and open cells and is self-expanding between positions defined by a compressed position and an expanded position, wherein the expanded position has a diameter of between about 16 to about 26 times the size of the diameter of the compressed position.

11. The self-expanding intrasaccular occlusion device of claim 10, wherein the single, contiguous superelastic shape-memory material comprises a nitinol scaffold laser cut from a single nitinol tube.

12. The self-expanding intrasaccular occlusion device of claim 11, wherein the expanded position of the scaffold is between at or about 1.5 mm to at or about 12 mm in diameter; and the compressed position is between about 0.25 mm to about 0.6 mm in diameter.

13. A system comprising the self-expanding intrasaccular occlusion device of claim 10, further comprising a guidewire and deployment anchor.

14. A method of making an intrasaccular occlusion device, comprising:
forming a single, contiguous superelastic shape-memory material into to a predefined pattern, the pattern defining a scaffold having zones of flexure and open cells;
processing the superelastic shape-memory material to comprise the scaffold having an orb-like shape memory; and
finishing the scaffold to be biocompatible.

15. The method of claim 14, wherein the material is nitinol.

16. The method of claim 15, wherein the nitinol comprises a single nitinol tube and the forming step comprises laser cutting the nitinol tube according to the predefined pattern.

17. The method of claim 14, wherein the single, contiguous superelastic shape-memory material is formed by additive manufacture.

18. The method of claim 17, wherein the material comprises nitinol.

19. The method of claim 14, wherein the zones of flexure and open cells are placed in a manner within the scaffold that when in an orb-like shape it is flexible, soft and compliant.

20. A method of treating an aneurysm, comprising:
positioning the device of claim 1 adjacent the neck of an aneurysm;
deploying the device within the sac of the aneurysm and permitting the device to self-expand within the sac; and
introducing an embolic material to the sac and within the device through one or more of the open cells.
